(19) United States
(12) Patent Application Publication
(10) Pub. No.: US 2017/0143278 A1
(43) Pub. Date: May 25, 2017

(54) OBJECT INFORMATION ACQUIRING APPARATUS AND SIGNAL PROCESSING METHOD

(71) Applicant: CANON KABUSHIKI KAISHA, Tokyo (JP)

(72) Inventor: Yoshiko Nakamura, Kyoto-shi (JP)

(21) Appl. No.: 15/304,907

(22) PCT Filed: May 8, 2015
(86) PCT No.: PCT/JP2015/063961

§ 371 Date: Oct. 18, 2016

(30) Foreign Application Priority Data
May 19, 2014 (JP) 2014-103640
Apr. 15, 2015 (JP) 2015-083523

(51) Int. Cl. A61B 5/00; A61B 5/145

(52) U.S. Cl. CPC A61B 5/7235; A61B 5/0095; A61B 5/14546; A61B 5/7278; A61B 5/742

(57) ABSTRACT

Provided is an object information acquiring apparatus including: a detector that detects an acoustic wave which is generated from an object irradiated by light with a first wavelength and light with a second wavelength, and outputs an electric signal; a processor that determines a weight coefficient in accordance with concentration of a specified substance inside the object in use of a first signal, which is an electric signal derived from the light with the first wavelength, and a second signal, which is an electric signal derived from the light with the second wavelength, and weights the electric signal outputted from the detector; and a generator that generates specific information inside the object based on the weighted electric signals.

### TECHNICAL FIELD

The present invention relates to an object information acquiring apparatus and a signal processing method.

### BACKGROUND ART

As an apparatus that images inside an inspection target (object) using an ultrasound wave (acoustic wave), a photoacoustic tomography (PAT) apparatus used for medical diagnosis, for example, has been proposed. The photoacoustic tomography apparatus irradiates a laser pulsed light into an object, receives a photoacoustic wave, which is generated as a result of a tissue in the object absorbing the energy of the irradiated light, using a probe, and generates an image in accordance with the optical characteristic values inside the object.

However the photoacoustic waves that the probe receives includes not only the photoacoustic waves from the tissue of interest, but also the photoacoustic waves generated from other biological tissues (e.g. skin). These photoacoustic waves cause a drop in contrast of the region of interest.

Japanese Patent Application Laid-open No. 2013-055988 (PTL1) discloses a photoacoustic tomography apparatus that deletes signals generated from the skin. In Japanese Patent Application Laid-open No. 2013-055988 (PTL1), light with a first wavelength that is easily absorbed by the skin and light with a second wavelength that is hardly absorbed by the skin and reaches inside the biological tissue are irradiated into the object. Then an image, in which the influence of the skin is reduced, is created by subtracting a signal (or image) derived from the first wavelength from a signal (or image) derived from the second wavelength.

### CITATION LIST

### Patent Literature

PTL1: Japanese Patent Application Laid-open No. 2013-055988

### SUMMARY OF INVENTION

### Technical Problem

The apparatus according to Japanese Patent Application Laid-open No. 2013-055988 (PTL1) acquires an acoustic wave using light with a first wavelength which can easily be absorbed by skin. However if a tissue of interest is located immediately under the skin, even light with the first wave, which can easily be absorbed by skin, may reach the tissue of interest, and a photoacoustic wave may be generated from the tissue of interest. In such a case, if a signal that is measured using light with the first wavelength is determined as a skin signal, and is subtracted from a signal that is measured using light with a second wavelength, a signal of the tissue of interest located immediately under the skin may be deleted as well. As a result, the acquired contrast of the region of interest may drop.

The present invention is based on such a problem recognition described above. It is an object of the present invention is to provide a technique to enhance an image of the tissue of interest to photoacoustic tomography.

### Solution to Problem

The present invention provides an object information acquiring apparatus, comprising:

a light source that can irradiate at least light with a first wavelength and a light with a second wavelength;

a detector that detects an acoustic wave generated from an object into which light is irradiated from the light source, and outputs an electric signal;

a processor that determines a weight coefficient in accordance with concentration of a specified substance inside the object in use of a first signal, which is an electric signal derived from the light with the first wavelength, and a second signal, which is an electric signal derived from the light with the second wavelength, and weights the electric signal outputted from the detector in use of the weight coefficient in accordance with the concentration of the specified substance; and

a generator that generates image data indicating specific information inside the object based on the electric signal weighted by the processor.

Further, the present invention also provides an object information acquiring apparatus, comprising:

a light source that can irradiate at least light with a first wavelength and light with a second wavelength;

a detector that detects an acoustic wave generated from an object into which light is irradiated from the light source, and outputs an electric signal;

a generator that generates first specific information inside the object based on an electric signal derived from the light with the first wavelength, and second specific information inside the object based on an electric signal derived from the light with the second wavelength; and

a processor that determines a weight coefficient in accordance with concentration of a specified substance inside the object in use of the first specific information, the second specific information and absorption coefficients of the specified substance at the respective wavelengths, and weights image data indicating the specific information inside the object in use of the weight coefficient based on the concentration of the specified substance.

Further, the present invention also provides a signal processing method for electric signals based on acoustic waves generated from an object irradiated by light with a first wavelength and light with a second wavelength,

the method comprising:

a step of determining a weight coefficient in accordance with concentration of a specified substance inside the object in use of a first signal, which is an electric signal derived from the light with the first wavelength, and a second signal, which is an electric signal derived from the second wavelength;

a step of weighting an electric signal outputted from the detector in use of a weight coefficient in accordance with the concentration of the specified substance; and

a step of generating an image indicating specific information inside the object based on the weighted electric signals.

Further, the present invention also provides a signal processing method for electric signals based on acoustic waves generated from an object irradiated by light with a first wavelength and light with a second wavelength,

the method comprising:

a step of generating first specific information inside the object based on an electric signal derived from the light with the first wavelength;

a step of generating second specific information inside the object based on an electric signal derived from the light with the second wavelength;

a step of determining a weight coefficient in accordance with concentration of a specified substance inside the object in use of the first specific information, the second specific information and absorption coefficients of the specified substance at the respect wavelengths; and

a step of weighting an image which includes the specific information inside the object in use of a weight coefficient based on the concentration of the specified substance.

### Advantageous Effects of Invention

According to the present invention, a technique to enhance an image of the tissue of interest can be provided to photoacoustic tomography.

Further features of the present invention will become apparent from the following description of exemplary embodiments with reference to the attached drawings.

### BRIEF DESCRIPTION OF DRAWINGS

FIG. 1 is a schematic diagram depicting a configuration of a photoacoustic tomography apparatus;

FIG. 2 is an absorption coefficient spectral diagram of an absorber;

FIG. 3 is an absorption spectral diagram of signals calculated by the photoacoustic tomography apparatus;

FIGS. 4A and 4B are set of images showing the effect of the photoacoustic tomography apparatus according to Example 1;

FIG. 5 is a schematic diagram depicting a configuration of a photoacoustic tomography apparatus according to Example 3;

FIG. 6A is an image showing an effect of the photoacoustic tomography apparatus according to Example 3;

FIG. 6B is another image showing an effect of the photoacoustic tomography apparatus according to Example 3;

FIG. 6C is another image showing an effect of the photoacoustic tomography apparatus according to Example 3; and

FIG. 7 is a flow chart depicting the signal processing.

### DESCRIPTION OF EMBODIMENTS

Embodiments of the present invention will now be described with reference to the drawings. The dimensions, materials, shapes or the like of the components described herein below and the relative arrangement thereof should be appropriately changed depending on the configuration and various conditions of the apparatus to which the present invention is applied, and the scope of the present invention should not be limited to the following description.

The present invention relates to a technique to detect an acoustic wave propagated from an object, and to generate and acquire specific information inside the object. Therefore the present invention can be understood as an object information acquiring apparatus, a control method thereof, an object information acquiring method, and a signal processing method. The present invention can also be understood as a program that causes an information processor, which includes such a hardware resource as a CPU, to execute these methods, and a storage medium storing the program.

The object information acquiring apparatus of the present invention includes an apparatus that irradiates light (electromagnetic wave) into an object, and receives (detects) an acoustic wave which is generated in a specific position inside the object or on the surface of the object, and propagates by the photoacoustic effect, utilizing a photoacoustic tomography technique. This object information acquiring apparatus, which acquires specific information inside the object in the format of image data based on photoacoustic measurement, can be called a “photoacoustic imaging apparatus” or a “photoacoustic image-forming apparatus”. This object information acquiring apparatus may also be called a “photoacoustic tomography apparatus”.

The specific information in the photoacoustic apparatus indicates a generation source of an acoustic wave generated by light irradiation, initial sound pressure inside the object, light energy absorption density and absorption coefficient derived from the initial sound pressure, or concentration of a substance constituting the tissue. In concrete terms, concentration of a substance concerns a blood component, such as the concentration of oxy-/dioxy-hemoglobin and oxygen saturation determined there from, or lipids, collagen, water or the like. The specific information may be determined as distribution information that indicates numeric data at each position inside the object. In other words, the distribution information, such as absorption coefficient distribution and oxygen saturation distribution, may be used as the object information.

The “acoustic wave” referred to in the present invention is typically an ultrasound wave, and includes an elastic wave called a “sound wave” and an “acoustic wave”. An acoustic wave generated by the photoacoustic effect is called a “photoacoustic wave” or “light-induced ultrasound wave”. An electric signal, which is converted from an acoustic wave by a probe, is also called an “acoustic signal”.

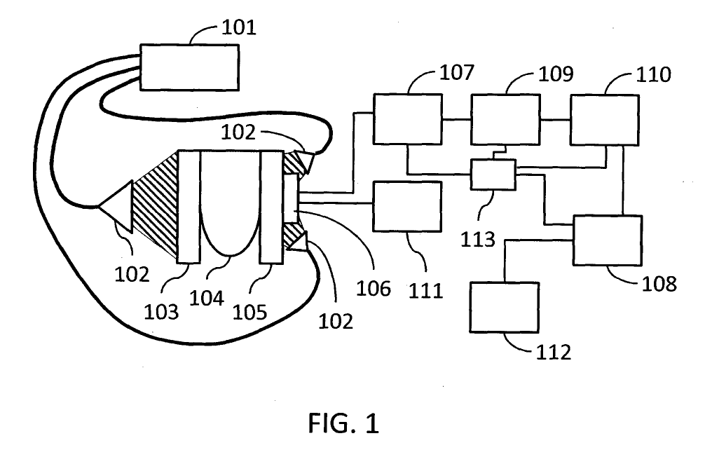

In FIG. 1, the photoacoustic tomography apparatus includes a light source 101, a light irradiator 102, a holding member 103, an acoustic matching member 105, a probe 106, a probe scanning driver 111, and a scanning/signal acquisition controller (not illustrated). The photoacoustic tomography apparatus further includes a signal processor 107, a reconstructor 108, a spectral analysis processor 109, an unnecessary signal processor 110, a display 112, and a signal recording memory 113. A measurement target is an object 104, such as a breast of a human body.

The light source 101 generates a pulsed light having a first wavelength, and guides the pulsed light to the light irradiator 102 via a bundle fiber. The light irradiator 102 irradiates the pulsed light into the object via the holding member 103. An absorber in the object 104 generates a photoacoustic wave by the pulsed light irradiated into the object. This photoacoustic wave is propagated through the acoustic matching member 105 and is received by the probe 106. The probe 106 scans along a plane or a spherical surface by the probe scanning driver 111. The scanning of the probe and timing of the signal acquisition are controlled by the scanning/signal acquisition controller (not illustrated).

The probe 106 converts the received photoacoustic wave into an analog electric signal. The signal processor 107 performs digital conversion and amplification for the analog electric signal, and records the digital electric signal in the memory. When scanning is over, the signal processor 107 averages the signals acquired at a same position, and outputs the result. The signal memory 113 records the signal with the first wavelength outputted from the signal processor 107.

The light source 101 can also irradiate a pulsed light having a second wavelength, which is different from the first wavelength. In the same manner as the case of the first wavelength, the signal processor 107 receives a photoacoustic wave from the object, and outputs an electric signal. The signal derived from the second wavelength is processed by the signal processor 107, and is recorded in the signal memory 113. The electric signals derived from the light with the first wavelength and the light with the second wavelength correspond to the first signal and the second signal of the present invention respectively. In the case of a light source that can irradiate lights with three or more wavelengths as well, a third signal derived from the light with the third wavelength is acquired.

The spatial analysis processor 109 reads out signals derived from each of the wavelengths from the signal memory 113, analyzes and compares the signal intensity and the constituent spectrum of the breast between the two wavelengths, estimates the concentration of the constituent specified in advance (specified substance), and, creates concentration distribution data of the constituent. The “constituent specified in advance” here refers to a constituent that is unnecessary for diagnosis in an image. Based on the concentration distribution data of the constituent specified by the spectral analysis processor 109, the unnecessary signal processor 110 performs reducing processing on the signal components that are estimated to be generated from this constituent, and outputs the result. The reconstructor 108 performs reconstruction or the like on the output signals from the unnecessary signal processor 110, and creates an absorption coefficient distribution in the object. Then the display 112 displays the absorption coefficient distribution. The spectral analysis processor and the unnecessary signal reducer correspond to the processor of the present invention.

For the light source, a light source that generates a nano-second order pulsed light with at least two wavelengths is used. The wavelength of the pulsed light is preferably 700 nm or more. If the wavelength of the light is less than 700 nm, the light is easily absorbed by hemoglobin, collagen or the like, and cannot reach a sufficient depth in the object. The wavelength of the pulsed light is normally changed depending on the constituent, since the light absorption spectrum is different depending on the constituent. The absorption spectrum is an absorption coefficient of a constituent (specified substance) at a plurality of wavelengths.

By using a wavelength at which the absorption spectrum of the constituent is distinctive, the accuracy to reduce signals improves. For example, if a plurality of wavelengths is selected, it is preferable that the absorption spectral shape of the constituent in the object is distinctively different at each of the wavelengths. To accurately calculate the concentration of the constituent even more so, it is preferable to find the distinctive extreme values of a peak or null (trough) in the absorption spectrum of the constituent, and select a plurality of wavelengths around these extreme values. In the processing according to the present invention, the unnecessary signals may be completely removed to zero, but the unnecessary signal reducing processing may only be to a degree, in which sufficient visibility for diagnosis is guaranteed.

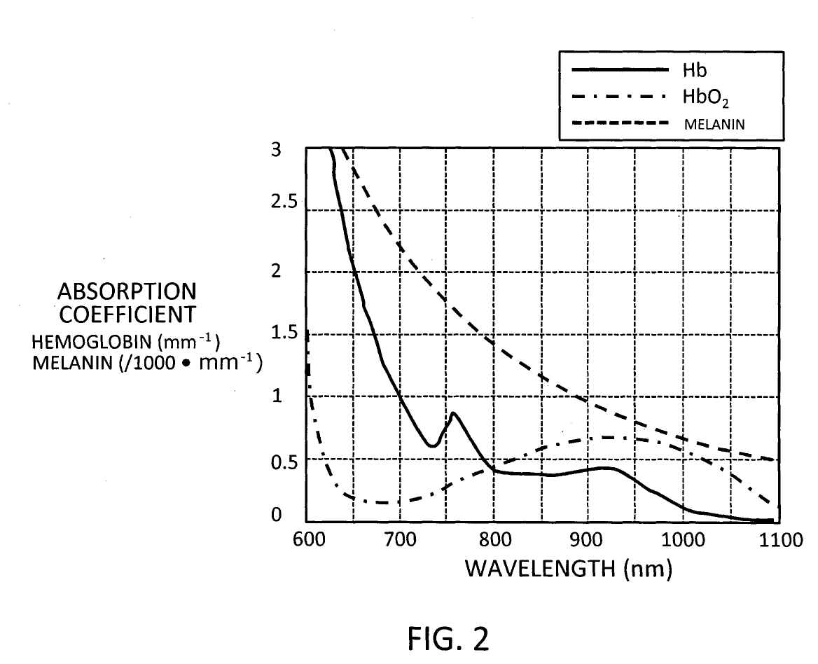

In this example, the skin (containing melanin) and the blood (containing hemoglobin) of the object are selected as the measurement target constituents. FIG. 2 shows a spectrum of each constituent. The absorption spectral shape of dioxy-hemoglobin (Hb) includes a distinctive null (through) and peak in the 700 nm to 790 nm wavelength range. Therefore it is preferable to select a plurality of wavelengths in this range. When the wavelength exceeds about 925 nm, the spectral intensity decreases in all the constituents. Hence if two wavelengths: a wavelength that is 925 nm or more, and a wavelength in the 700 nm to 925 nm are selected, the shapes of the spectra become similar. Therefore it is preferable to select two or more wavelengths in the 700 nm to 925 nm wavelength range. In this embodiment, five wavelengths: 740 nm, 760 nm, 800 nm, 825 nm and 850 nm, are selected.

A laser is preferable to acquire high power, but a light emitting diode or the like may be used instead of the laser. For the laser, various lasers, including a solid-state laser, a gas laser, a dye laser and a semiconductor laser may be used. Irradiation timing, waveform, intensity or the like are controlled by a light source controller, which is not illustrated.

In order to guide the light from the light source to the object, an optical member such as a mirror that reflects light, a lens that collects, expands or changes the shape of light, a prism that disperses, refracts or reflects light, an optical fiber that propagates light, or a diffusion plate may be used.

The probe 106 is a detector that has one or more elements for receiving an acoustic wave (ultrasound wave). If the probe 106 is a type where a plurality of elements is arranged on a plane, signals can be acquired all at once from a plurality of positions. Thereby the receiving time can be decreased, and the influence by vibration of the object or the like can be reduced. The probe receives and amplifies the acoustic wave, converts the wave into an electric signal, and outputs the electric signal. The elements used for the probe are, for example, conversion elements using a piezoelectric phenomenon, conversion elements using the resonance of light, and conversion elements using the change in capacitance. The configuration of the elements is not restricted if only an acoustic wave can be received and converted into an electric signal. The probe or the elements corresponds to the detector of the present invention.

The probe scanning driver 111 scans the probe 106. By scanning the probe 106, the photoacoustic waves can be acquired over a wide range of the object. The scanning/signal acquisition controller (not illustrated) controls the scanning timing and scanning pitch. The timing to acquire a signal is measured synchronizing with the light source controller. A scanning range where the probe 106 is moved by the probe scanning driver 111, a number of times of averaging signals or the like are recorded in memory (not illustrated), and can be externally changed by the operator. When estimating concentration of the constituents by analyzing signal intensities as this embodiment, it is desirable that the probe scanning driver 111 scans the probe 106 so as to be arranged on a same position and face a same direction. Here, the same position means a distance narrower than half of the resolution of the apparatus, and the same direction means an angular difference smaller than half of the directivity angle of the probe 106.

The electric signals are inputted from the probe 106 to the signal processor 107. The signal processor 107 amplifies the electric signals and performs analog-digital conversion. Before or after the amplification or analog-digital conversion, the signal processor 107 may add and average the electric signals acquired by the probe scanning at a same position. The output of the signal processor 107 is recorded in the signal memory 113.

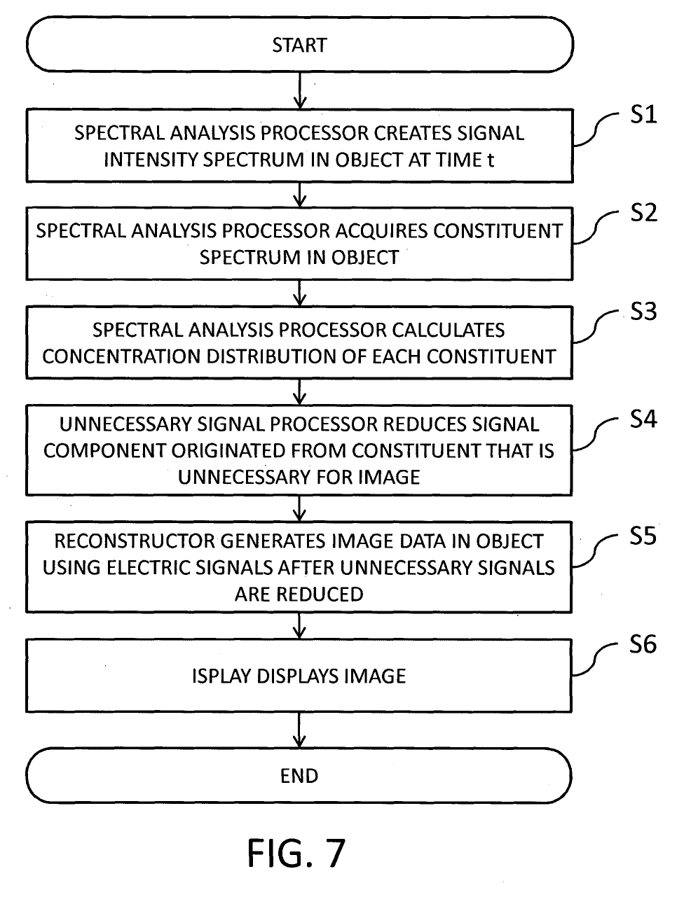

An example of the processing flow of time series signals recorded in the signal memory 113 will be described with reference to FIG. 7. The spectral analysis processor 109 reads out the measured time series signals with a plurality of wavelengths from the signal memory 113. Signals relating to the wavelength which had been measured lastly may be directly inputted to the spectral analysis processor 109 instead of being recorded in the signal memory 113.

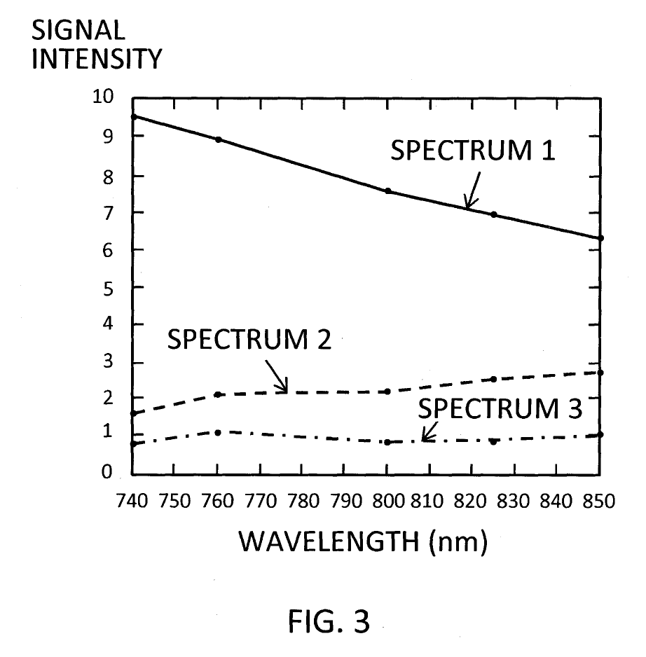

The spectral analysis processor 109 creates a signal intensity spectrum at time t out of the time series signals (step S1). For example, FIG. 3 shows the signal intensity spectrum at three locations in the object, created based on the time series signals acquired by lights with five wavelengths (740 nm, 760 nm, 800 nm, 825 nm and 850 nm). A spectrum 1 is a spectrum acquired from the intensity of the received signal corresponding to a photoacoustic wave acquired in a segment close to the surface. A spectrum 2 corresponds to a segment that is deeper than the case of the spectrum 1, and a spectrum 3 corresponds to a segment that is even deeper than the case of the spectrum 2. FIG. 3 shows the intensity of a signal (generated after the light is irradiated) that is acquired at a predetermined time when the signal has sufficient intensity, and is a graph acquired by connecting the intensity value at each wavelength. In FIG. 3, the abscissa indicates the wavelength, and the ordinate indicates the signal intensity (normalized relative value).

The spectral analysis processor 109 acquires the constituent spectra inside the breast (step S2). It is assumed that a number of constituents in the object is k=1, . . . , N, and the spectra are known. The types of the constituents and the spectra thereof are stored in memory (not illustrated) in the spectral analysis processor. The types of the constituents and the spectra thereof can be overwritten, and the constituent types used for the following spectral analysis processing can be selected from the constituents recorded in memory.

Then the spectral analysis processor 109 calculates the concentration of a specified substance (constituent k) and a distribution thereof (step S3). The wavelength λ of light that is irradiated into the object for the i-th time is λi. When the light with wavelength λᵢ is irradiated into the object and a photoacoustic wave is emitted from a constituent located at a position rₒ inside the object at time tₒ, the sound pressure P, which the probe located at the position r receives at time t, is given by the following Expression (1).

$$\begin{matrix}[ Math . 1 ] & \\ P ( λ i , r , t ) = \int {\sum }_{k = 1}^{N} φ ( {λ}_{i} , {r}_{0} ) \cdot {C}_{k} ( {r}_{0} ) \cdot μ {a}_{k} ( λ i ) \cdot G ( r , t \vert {r}_{0} ) d {r}_{0} & ( 1 )\end{matrix}$$

Here Φ denotes the light quantity distribution inside the object, Cₖ denotes a concentration of the constituent k, μaₖ denotes an absorption coefficient of the constituent k, and G denotes a Green function which indicates the propagation of an acoustic wave generated at the position rₒ until entering the probe. The Green function indicates probe characteristics, such as the response and directivity of the probe, and physical characteristics, such as the attenuation of the ultrasound wave and the generation form of the photoacoustic wave. The absorption coefficient μaₖ and the light quantity distribution inside the object Φ change depending on the wavelength λᵢ of the irradiated light.

If the light quantity distribution Φ is normalized, Expression (1) becomes the following Expression (2).

$$\begin{matrix}[ Math . 2 ] & \\ P ( λ i , r , t ) = {\sum }_{k = 1}^{N} μ {a}_{k} ( λ i ) {A}_{k} ( r , t ) where \\ {A}_{k} ( r , t ) = \int {C}_{k} ( {r}_{0} ) \cdot G ( r , t \vert {r}_{0} ) d {r}_{0} & ( 2 )\end{matrix}$$

Aₖ is a coefficient that is determined by the concentration of the constituent k and the Green function that indicates the propagation to the probe. Aₖ is also a function of the concentration of the constituent k, and the value of Aₖ increases as the concentration of the constituent increases. The concentration of the constituent k and the Green function are functions that do not depend on the wavelength, hence Aₖ is also a function that does not depend on the wavelength. If Aₖ is known, the concentration ratio of the constituent in the object can be indirectly estimated.

Expression (2) indicates that the amplitude value spectrum of the signal is the sum of the absorption coefficient spectra of a plurality of constituents multiplied by weight Aₖ which does not depend on the wavelength. Therefore the weight coefficient Aₖ of each constituent can be determined by fitting the amplitude spectrum of the signal using the absorption spectrum of each constituent. A common method that is used for fitting is the least squares method. According to the least squares method, the weight coefficient Aₖ, that satisfies the conditions of the following Expression (3), is determined.

$$\begin{matrix}[ Math . 3 ] & \\ {\hat{A}}_{k} ( r , t ) = arg {min}_{( Ak )} {\vert P ( λ i , r , t ) - {\sum }_{k = 1}^{N} {A}_{k} ( r , t ) \cdot μ {a}_{k} ( λ i ) \vert}^{2} & ( 3 )\end{matrix}$$

The fitting method is not limited to the least squares method, and a commonly used maximum likelihood method, expected value maximization method or the like may be used instead. It is desirable to change the fitting method depending on the type of noise that is generated in the sound pressure P. If the noise has a Gaussian distribution, using the least squares method is preferable. If the noise has a distribution that is different from Gaussian distribution, such as a case of Poisson distribution or Rayleigh distribution, then using the maximum likelihood method in accordance with the type of noise is preferable. To uniquely determine the weight coefficient Aₖ to be estimated, it is preferable that a number of wavelengths is greater than a number of weight coefficients. However if the number of weight coefficients Aₖ to be estimated is greater than the number of wavelengths, the value of the weight coefficient Aₖ can be uniquely determined by adding other conditions to Expression (3). Conditions other than Expression (3) are, for example, a condition for the weight coefficient Aₖ to smoothly change at adjacent timings, or a condition for the weight coefficient Aₖ to have a minimum value. The characteristics of the photoacoustic signal and the characteristics of the object may be included in the conditions. In Examples 1 and 2, coefficients A of three constituents are estimated from data on two wavelengths. In the examples, a condition for the weight coefficient Aₖ to have a minimum value has been added.

The coefficient of the constituent k is as follows.

Âₖ [Math 4]

The spatial analysis processor 109 outputs the distribution of this coefficient to the unnecessary signal processor 110. The distribution information of the weight coefficient Aₖ is inputted to the unnecessary signal processor 110 from the spectral analysis processor 109, and the acquired signal is inputted to the unnecessary signal processor 110 from the signal memory 113. The unnecessary signal processor 110 reduces a signal of a constituent that is unnecessary for an inspection image that is set in advance by the operator or within the apparatus (step S4). For example, the signal components are reduced using the distribution of the weight coefficient Aₖ of the unnecessary constituent k, as shown in the following Expression (4).

[Math. 5]

S(λi,t)=P(λi,t)−Âₖ(t)·μαₖ(λi) (4)

Here S is a signal after the unnecessary constituents are reduced. According to Expression (4), the spectral analysis processor 109 acquires a weight coefficient Aₖ by which the reducing amount increases as the concentration of the constituent k increases.

For the reducing method, signals of which weight coefficient Aₖ, in accordance with the concentration of the constituent k, is higher than a predetermined threshold may be reduced. In other words, a signal by a constituent, of which concentration is higher than a predetermined threshold, may be selectively reduced.

In the case of the two wavelengths measurement, not only performing the above processing method but also the ratio of the signals having the two wavelengths may be calculated, and the signals of the unnecessary constituents may be reduced based on this ratio. The ratio of the signals having the two wavelengths is determined by the spectral analysis processor according to Expression (5).

$$\begin{matrix}[ Math . 6 ] & \\ \frac{P ( {λ}_{1} , t )}{P ( {λ}_{2} , t )} = \frac{{\sum }_{k = 1}^{N} {A}_{k} ( t ) μ {a}_{k} ( {λ}_{1} )}{{\sum }_{k = 1}^{N} {A}_{k} ( t ) μ {a}_{k} ( {λ}_{2} )} & ( 5 )\end{matrix}$$

If it is assumed that the signal by the constituent k is high at time t and signals by constituents other than constituent k can be ignored, then Expression (5) becomes Expression (6).

$$\begin{matrix}[ Math . 7 ] & \\ \frac{P ( {λ}_{1} , t )}{P ( {λ}_{2} , t )} = \frac{μ {a}_{k} ( {λ}_{1} )}{μ {a}_{k} ( {λ}_{2} )} & ( 6 )\end{matrix}$$

Therefore the spectral analysis processor 109 estimates that a large number of constituents k are included at time t when the ratio of the signals having the two wavelengths is close to the absorption coefficient ratio of the constituents k, and calculates the distribution of the constituent k at time t. Then the unnecessary signal reducer reduces the signals at time t based on this estimated distribution value.

A time series electric signal in which signal on time t has been reduced is inputted to the reconstructor 108 from the unnecessary signal processor 110. The reconstructor 108 reconstructs an image using the inputted signal, and generates image data that indicates the absorption coefficient distribution, which is specific information inside the object (step S5). As the reconstruction method, universal back projection, which is used as a tomography technique, is used in the present invention. Other reconstruction methods are, for example, reverse projection in the Fourier domain, aperture synthesis, and a time reversal method. The reconstructor corresponds to the generator of the present invention.

The reconstructor 108 allows the display to display the reconstructed image (step S6). A possible display method is to use an MIP (Maximum Intensity Projection) image or a slice image. A method of displaying a 3D image in a plurality of directions may also be used. Further, the user may change the inclination, display region, window level or window width of the display image while checking the display. It is preferable that the reconstructor 108 allows the display to display images before and after performing the unnecessary signal reducing processing and the differential image thereof on the display. To compare the images before and after the reducing processing, it is preferable to display the images in small windows.

According to this embodiment, an image that indicates specific information inside the object can be generated based on the signals after the components of the unnecessary constituents k are reduced. In other words, an image in which the region of interest, other than the unnecessary constituents k, is enhanced, can be generated.

Weighting may be performed for the time series signals which are acquired by irradiating light at a timing that is different from the timing of acquiring the signal intensity spectrum. In this case, it is preferable to acquire the time series signals in the same measurement state as the case of acquiring the signal intensity spectrum. In this case as well, weighting for enhancing the image of the tissue of interest can be performed, since concentration is similar in the object.

<Modifications>

The photoacoustic tomography apparatus for reducing unnecessary signals based on the signal intensities has been described thus far. Here, the reducing of unnecessary signals of target constituents may be performed based on a concentration of the target constituents which has been obtained by the spectral analysis processing executed by the spectral analyzing processor 109 using the specific information distribution (image).

When the image intensity is used, the light quantity distribution Φ is normalized, as shown in Expression (7).

$$\begin{matrix}[ Math . 8 ] & \\ I ( λ i , {r}_{0} ) = {\sum }_{k = 1}^{N} {A}_{k} ( {r}_{0} ) \cdot μ {a}_{k} ( λ i ) & ( 7 )\end{matrix}$$

rₒ is a position inside the object, that is, a voxel position in the image. According to the comparison of Expression (7) and Expression (2), the weight coefficient Aₖ is the function of time t and element position r in the case of Expression (2), but is the function of the voxel position rₒ in the image in the case of Expression (7). However, it is the same that the image intensity is determined by the weight coefficient Aₖ, which does not change depending on the absorption coefficient and the wavelength. By replacing the time and the element position in Expression (3) with the voxel position in the image, the spectral analysis processor can compute Expression (7) in the same manner as Expression (3). In this case, the first specific information and the second specific information are generated from the electric signals derived from the light with the first wavelength and the electric signals derived from the light with the second wavelength respectively, and the components of a specific constituent (specified substance) are reduced from the image that indicates the specific information.

In this embodiment, the target is the constituent inside the object, but medicine contained in the object and a molecular probe can also be handled as a constituent of the present invention.

The coefficient Aₖ is a function of concentration. Therefore if oxy-hemoglobin or deoxy-hemoglobin is contained in the constituent as in the case of this example, oxygen saturation can be calculated using the two constituent coefficients A calculated by the spectral analysis processor. If glucose and water are the constituents, blood sugar concentration can be calculated.

According to this embodiment, weighting can be performed for images indicating the first specific information and the second specific information received when the signal intensity spectrum is acquired, or for imagines indicating new specific information acquired from this information.

Weighting may be performed for an image received by irradiating light at a timing that is different from the timing when the signal intensity spectrum is acquired. Weighting may also be performed for an image indicating the concentration distribution of a substance acquired from the signals corresponding to a plurality of wavelengths. In these cases as well, weighting to enhance an image of the tissue of interest can be performed if it is assumed that the object has a concentration similar to when the signal spectrum was acquired.

According to this embodiment, an image indicating the specific information inside the object, where components of unnecessary constituents k have been reduced, can be generated. In other words, an image where the region of interest, other than the unnecessary constituents k, is enhanced, can be generated.

In the above embodiment, an example of performing weighting to reduce the components of the unnecessary constituents was described, but the present invention is not limited to this method as long as the weighting can enhance the image of the tissue of interest. For example, the image of the tissue of interest may be enhanced by performing the weighting to amplify the components of the constituents of the tissue interest more than the components of the unnecessary constituents.

### EXAMPLE 1

An example of the object information acquiring apparatus according to the present invention will be described. This example will be explained using the apparatus shown in FIG. 1.

The light source 101 is a titanium sapphire laser. For the wavelengths of the light to be irradiated, two wavelengths: about 760 nm and about 800 nm, are selected. In other words, wavelengths of at least 700 nm, which reach a deep are in the object, are used for the measurement in this example. As the diagram of the absorption spectrum in FIG. 2 shows, the inclination of the absorption spectrum of deoxy-hemoglobin and that of oxy-hemoglobin are different at wavelengths 760 nm and 800 nm. In a wavelength around 800 nm, the absorption spectrum of the deoxy-hemoglobin and that of oxy-hemoglobin (HbO₂) cross. The quantity of light of each wavelength 760 nm and 800 nm is 57 mJ and 54 mJ respectively. To excite the titanium sapphire laser, an Nd-YAG laser light (pulsed light in the nano-second order at wavelength 1064 nm) is used.

In a state of holding the object 104 between the holding member 103 and the acoustic matching member 105, a pulsed light was irradiated from the light source. The probe 106 received the photoacoustic wave, which was irradiated from the object and propagated by the photoacoustic effect, via the acoustic matching member 105. The probe has a 1 mm element width, a 1 mm element pitch, a 20 mm×30 mm size, and a 2 MHz central frequency. The probe scanning driver moved the probe at the timings of the light irradiation and the photoacoustic wave reception. The moving pitch is 1 mm horizontally and 10 mm vertically. Thereby a photoacoustic wave was acquired in a 150 mm×90 mm measurement range.

The signal processor 107 preformed averaging processing on the electric signals converted from the photoacoustic wave, and stored the respective signal with each wavelength in the recording memory 113.

In this example, the target to reduce the intensity is a signal from the melanin contained in the skin. First the spectral analysis processor 109 normalized this signal with each wavelength by the irradiation intensity of each wavelength. Then the estimated weight coefficient of the constituent of the normalized signal at time t was calculated using Expression (8).

$$\begin{matrix}[ Math . 9 ] & \\ = 1 - \vert \frac{μ {a}_{k} ( {λ}_{1} )}{μ {a}_{k} ( {λ}_{2} )} - \frac{P ( {λ}_{1} , t )}{P ( {λ}_{2} , t )} \vert / \frac{μ {a}_{k} ( {λ}_{1} )}{μ {a}_{k} ( {λ}_{2} )} & ( 8 )\end{matrix}$$

Here μaₖ is an absorption coefficient of melanin.

The unnecessary signal reducer 110 set the time when the normalized weight coefficient Aₖ is 0.8 to 1 as time T when a number of signals from melanin is high. The unnecessary signal reducer created signals by reducing the signal amplitude at time T in proportion to the concentration.

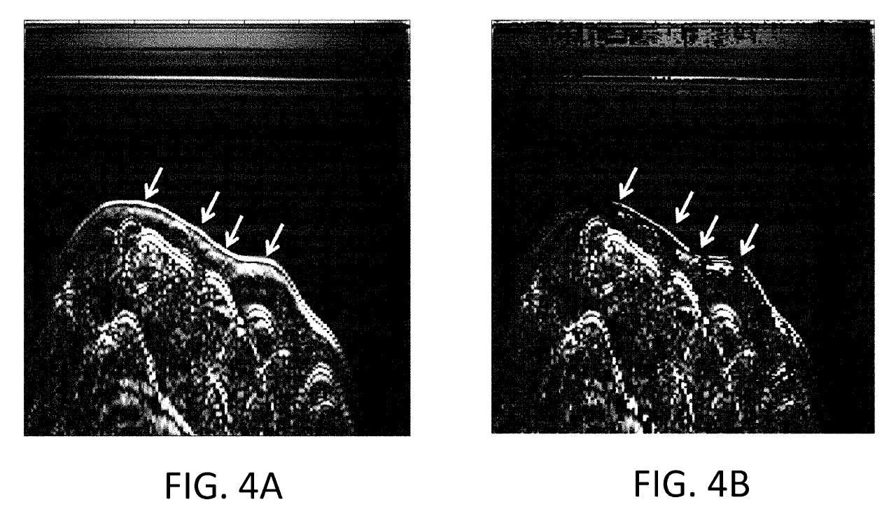

FIG. 4 shows the result. FIG. 4A shows signals before reducing the unnecessary signal, and FIG. 4B shows signals after reducing signals of melanin by applying the present invention. A white arrow indicates a position of a skin signal. In FIG. 4B, the skin signals have been reduced. Therefore if the object information acquiring apparatus of this example is used, signals derived from melanin, which is abundantly contained in the skin, can be reduced. The signals of the region of interest immediately under the skin were not reduced very much. In other words, according to this example, signals derived from areas, other than the regin of interest (tissue of interest), can be selectively reduced while suppressing a drop in contrast.

### EXAMPLE 2

In Example 1, the weight coefficient of the constituent is estimated using the ratio between wavelengths. In Example 2, the weight coefficient of the constituent is estimated using the signal intensity spectrum, and the signals from melanin of the skin are reduced. Mainly the differences from Example 1 will be described. This example will be explained using the apparatus shown in FIG. 1.

In this example as well, two wavelengths: about 760 nm and about 800 nm, are used to acquire signals. As the constituents, three types: melanin, oxy-hemoglobin and deoxy-hemoglobin contained in skin, are tested. In other words, three types of constituents are tested with signals with two wavelengths. Hence the spectral analysis processor added a constraint, to minimize the concentration of each constituent, to Expression (3), and estimated the weight coefficient of the constituent.

First the spectral analysis processor 109 normalized the signal with each wavelength by the irradiation intensity of each wavelength. Then the weight coefficient distribution of each constituent of the normalized signal at time t was calculated using Expression (3) and the constraint. The spectral analysis processor set the signal from melanin of the constituents of the object in the unnecessary signal reducer as the unnecessary signal, and inputted the calculated weight coefficient distribution of each constituent to the unnecessary signal reducer. The unnecessary signal reducer 110 normalized the signal read from the signal recording memory 113 by the irradiated light quantity, reduced the signals generated from melanin from the signals with a 800 nm wavelength using Expression (4), returned the signal intensity back to the intensity considering the irradiated light quantity, and outputted the signals. The reconstructor 108 received the input of the signals after reducing the signals from melanin, and created the absorption coefficient distribution image in the object.

When the image after reducing the unnecessary signals under these conditions was checked, it was found that components derived from the skin which contains melanin were reduced compared with the image before reducing the unnecessary signals.

### EXAMPLE 3

In Example 1 and 2, the constituent amount is estimated using the signal with two wavelengths. In Example 3, a case of estimating the constituent amount in the image, using a multi-wavelength image, will be described. In this example, a hemispherical (spherical crown type) probe is used. The correspondence between a number of wavelengths and the shape of the probe, however, is not limited to this, and various combinations can be used.

In this example, a phantom is measured using three wavelengths: 740 nm, 760 nm and 800 nm. The 740 nm wavelength is a wavelength of an area around null (through-shaped minimum value portion) of the deoxy-hemoglobin absorption spectrum. The 760 nm wavelength is the peak of the deoxy-hemoglobin absorption spectrum. And the 800 nm wavelength is a wavelength around the intersection of the absorption spectra of oxy-hemoglobin and deoxy-hemoglobin. Absorbers that stimulate the skin and blood vessels, of which oxygen saturation is 70% and 90% are disposed in the phantom.

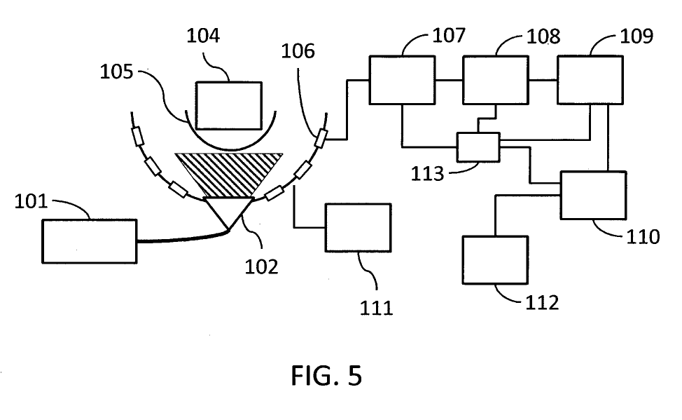

FIG. 5 is an overview of the configuration of the apparatus of this example. The correspondence of a reference numeral and a composing element is the same as FIG. 1. Water and the phantom are placed in a cup-type acoustic matching member 105 and measurement is performed. For the probe 106, 512 conversion elements (element width: 1 mm) are spirally disposed on the spherical surface (surface of the spherical crown). The center of the acoustic matching member and the center of the probe array are aligned. The radius of the probe array is 12.7 cm, and the radius of the acoustic matching material is 8 cm.

In this apparatus, the light source 101 irradiated light into the phantom, the probe 106 received the photoacoustic waves and outputted electric signals. The signal processor 107 processed the electric signals outputted from the probe 106 and recorded the processed signals into the signal recording memory 113. Then, the reconstructor 108 performed reconstructing using the signals read out from the signal recording memory 113, and recorded reconstructed image in the signal recording memory 113. Then, the measurement was continued while changing the wavelength. In this example, reconstructed images each relating to the plurality of wavelengths are stored in the signal recording memory 113. The spectral analysis processor 109 calculated the weight coefficient of each constituent by reading out the reconstructed images each relating to the plurality of wavelengths stored in the signal recording memory 113.

In this example, as the constituents, three types: melanin, oxy-hemoglobin and deoxy-hemoglobin contained in the skin, were tested. The spectral analysis processor calculated the weight coefficient of each constituent based on the least squares method, and inputted the coefficient distribution of each constituent to the unnecessary signal reducer 110. The unnecessary signal reducer 110 reduced the melanin image from the image based on the 760 nm wavelength using Expression (3), and outputted the result. The image after reduction, the image before reduction and the difference thereof were displayed.

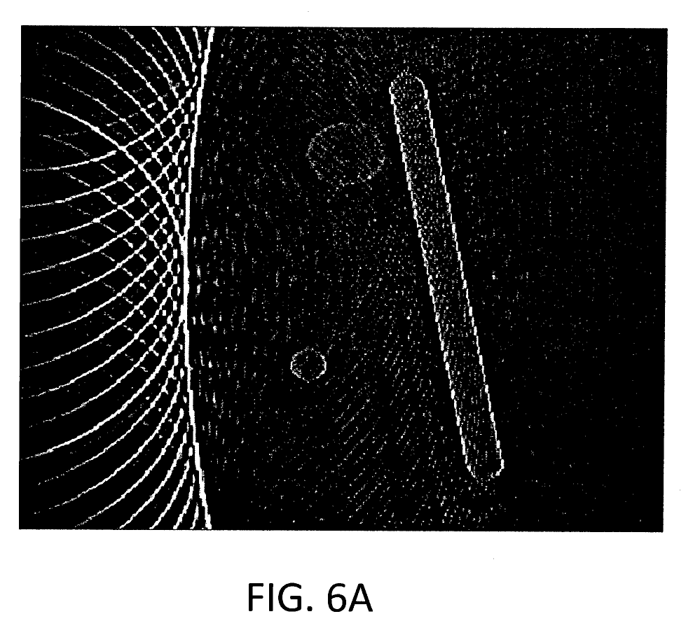

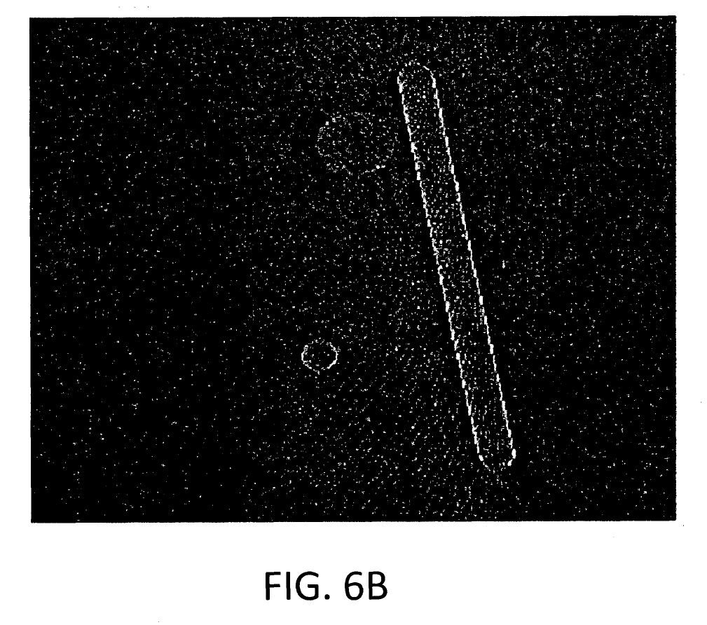

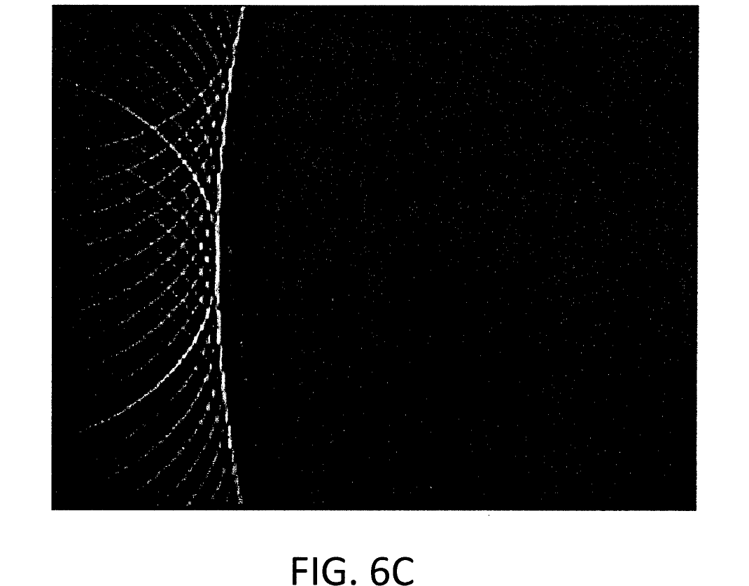

FIG. 6 shows the display result. FIG. 6A is an image before the reduction, and FIG. 6B is an image after the reduction. FIG. 6C is an image of the difference between FIG. 6A and FIG. 6B after the reduction. In FIG. 6A, the white meshed object seen at the left is an absorber having the absorption spectrum of melanin, and the rod-shaped object or the like at the right are the measurement targets, assuming a tissue containing blood exists. In FIG. 6B, the absorber at the left cannot be seen in the image. In this way, it was confirmed that the object information acquiring apparatus of this example can reduce the influence from skin by minimizing the effect on an image of the tissue in the region of interest.

As described in each example, according to the present invention, the influence of the photoacoustic wave generated from components that are not part of the tissue of interest or region of interest can be reduced in the stage of electric signals, or can be reduced in the stage of image data generated from the electric signals. The skin in particular is irradiated by strong light before attenuation, hence the strength of the signals generated from the skin is high. However if the influence of an acoustic wave derived from the skin can be reduced by the technique of the present invention, an excessive expansion of the dynamic range of the display image can be prevented, and the contrast of the image can be improved, whereby the present invention can contribute to good image diagnosis.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™ ) , a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-103640, filed on May 19, 2014, and, Japanese Patent Application No. 2015-083523, filed on Apr. 15, 2015 which are hereby incorporated by reference herein in their entirety.

1. An apparatus, comprising:
a light source that can irradiate at least light with a first wavelength and a light with a second wavelength;
a detector that detects an acoustic wave generated from an object into which light is irradiated from the light source, and outputs an electric signal;
a processor that determines a weight coefficient in accordance with concentration of a specified substance inside the object in use of a first signal, which is an electric signal derived from the light with the first wavelength, and a second signal, which is an electric signal derived from the light with the second wavelength, and weights the electric signal outputted from the detector in use of the weight coefficient in accordance with the concentration of the specified substance; and
a generator that generates image data indicating specific information inside the object based on the electric signal weighted by the processor.

2. An apparatus, comprising:
a light source that can irradiate at least light with a first wavelength and light with a second wavelength;
a detector that detects an acoustic wave generated from an object into which light is irradiated from the light source,
and outputs an electric signal;
a generator that generates first specific information inside the object based on an electric signal derived from the light with the first wavelength, and second specific information inside the object based on an electric signal derived from the light with the second wavelength;
and a processor that determines a weight coefficient in accordance with concentration of a specified substance inside the object in use of the first specific information, the second specific information and absorption coefficients of the specified substance at the respective wavelengths, and weights image data indicating the specific information inside the object in use of the weight coefficient based on the concentration of the specified substance.

3. The apparatus according to claim 1, wherein the processor acquires the weight coefficient in accordance with the concentration of the specified substance by fitting an intensity of the first signal and an intensity of the second signal into an absorption spectrum that indicates an absorption coefficient of the specified substance.

4. The apparatus according to claim 3, wherein
the processor acquires the weight coefficient Ak by performing a fitting that satisfies the following expression,
where k denotes the specified substance, Ak denotes the weight coefficient of the specified substance k, P denotes the intensity of the electric signal, μak denotes the absorption
coefficient of the specified substance k, r denotes the position of the detector, t denotes the detection time of the electric signal, and Xi denotes the wavelength of the light
irradiated from the light source into the object for the i-th time:
$$P ( λ i , r , t ) = {\sum }_{k = 1}^{N} μ {a}_{k} ( λ i ) {A}_{k} ( r , t ) .$$

5. The apparatus according to claim 2, wherein the processor determines the weight coefficient in accordance with the concentration of the specified substance by fitting the intensity in the first specific information and the intensity in the second specific information into the absorption spectrum that indicates the absorption coefficient of the specified substance.

6. The apparatus according to claim 5, wherein the processor acquires the weight coefficient Ak by performing a fitting that satisfies the following expression, where k denotes the specified substance, Ak denotes the weight coefficient of the specified substance k, I denotes the intensity in the specific information, μak denotes the absorption coefficient of the specified substance k, r denotes the position in the specific information, and λi denotes the wavelength of the light irradiated from the light source into the object for the i-th time:
$$I ( λ i , {r}_{0} ) = {\sum }_{k = 1}^{N} {A}_{k} ( {r}_{0} ) \cdot μ {a}_{k} ( λ i ) .$$

7. The apparatus according to claim 3, wherein the processor changes a method used for the fitting in accordance with the number of wavelengths that can be irradiated by the light source and the number of the specified substances.

8. The apparatus according to claim 7, wherein the processor adds a condition regarding the weight coefficient and performs the fitting when the number of specified substances is greater than the number of wavelengths that can be irradiated by the light source.

9. The apparatus according to claim 3, wherein the processor performs the fitting using the least squares method, maximum likelihood method, or expected value maximization method.

10. The apparatus according to claim 1, wherein the processor determines the weight coefficient based on the intensity ratio of the first signal and the second signal.

11. The apparatus according to claim 2, wherein the processor determines the weight coefficient based on the intensity ratio of the first specific information and the second specific information.

12. The apparatus according to claim 1, wherein the processor determines the weight coefficient so as to reduce a component derived from the specified substance when the concentration is higher than a predetermined threshold.

13. The apparatus according to claim 1, wherein the specified substance is melanin.

14. The apparatus according to claim 1, wherein the generator allows a display to display image data before and after the weighting processing.

15. The apparatus according to claim 1, wherein the generator generates differential image data of the image data before and after the weighting processing, and allows a display to display the differential image data.

16. The apparatus according to claim 1, further comprising a display that displays image data generated by the generator.

17. A signal processing method for electric signals based on acoustic waves generated from an object irradiated by light with a first wavelength and light with a second wavelength, the method comprising:
a step of determining a weight coefficient in accordance with concentration of a specified substance inside the object in use of a first signal, which is an electric signal derived from the light with the first wavelength, and a second signal, which is an electric signal derived from the second wavelength;
a step of weighting an electric signal outputted from the detector in use of a weight coefficient in accordance with the concentration of the specified substance; and a step of generating an image indicating specific information inside the object based on the weighted electric signals.

18. A signal processing method for electric signals based on acoustic waves generated from an object irradiated by light with a first wavelength and light with a second wavelength, the method comprising:
a step of generating first specific information inside the object based on an electric signal derived from the light with the first wavelength;
a step of generating second specific information inside the object based on an electric signal derived from the light with the second wavelength;
a step of determining a weight coefficient in accordance with concentration of a specified substance inside the object in use of the first specific information, the second specific information and absorption coefficients of the specified substance at the respect wavelengths; and
a step of weighting an image which includes the specific information inside the object in use of a weight coefficient based on the concentration of the specified substance.
